(19) United States
(12) Patent Application Publication
(10) Pub. No.: US 2025/0313653 A1
(43) Pub. Date: Oct. 9, 2025

(54) GUEST/HOST INCLUSION COMPLEXES CONTAINING S-NITROSOGLUTATHIONE AND METHODS OF USE THEREOF

(71) Applicant: VIRGINIA COMMONWEALTH UNIVERSITY, Richmond, VA (US)

(72) Inventors: Xuewei WANG, Richmond, VA (US); Wuwei LI, Richmond, VA (US)

(21) Appl. No.: 18/864,055

(22) PCT Filed: May 23, 2023
(86) PCT No.: PCT/US2023/023177

§ 371 Date: Nov. 8, 2024

Related U.S. Application Data
(60) Provisional application No. 63/350,114, filed on Jun. 8, 2022.
(60) Provisional application No. 63/393,428, filed on Jul. 29, 2022.

(51) Int. Cl. C08B 37/16; A61K 9/00; A61K 47/69

(52) U.S. Cl. CPC C08B 37/0015; A61K 9/008; A61K 47/6951

(57) ABSTRACT

Provided herein are compositions containing guest/host inclusion complexes. In particular, each complex includes S-nitrosoglutathione (GSNO) as the guest and substituted or unsubstituted cyclodextrin as the host, such as alpha cyclodextrin. The compositions may be used as an antibacterial and antithrombotic agent in catheter lock solutions and to deliver nitric oxide to a subject in need thereof.

### FIELD OF THE INVENTION

The disclosure generally pertains to compositions comprising guest/host inclusion complexes and methods of use thereof. In particular, each complex includes guest S-nitrosoglutathione (GSNO) and host cyclodextrin.

### BACKGROUND

Nitric oxide is a multifunctional gaseous radical that plays pivotal roles in a wide range of physiological and pathophysiological processes, such as vasodilation, coagulation, inflammation, neurotransmission, host defense, and wound healing.¹⁻⁵ Inspired by the biological functions of endogenous NO, exogenous NO has been employed as a therapeutic agent for pulmonary, cardiovascular, neurological, and renal diseases associated with NO deficiencies.^{6,7} NO has also been released or generated from medical implants and dressings to protect against thrombosis, infection, and inflammation.⁸⁻¹⁰ (There are three main avenues of NO delivery. First, NO gas is inhaled into the respiratory system, which is an FDA-approved therapy for hypoxic respiratory failure associated with pulmonary hypertension.¹¹ Second, solid NO donors are embedded in nanoparticles and polymers, allowing for controlled release of NO from storable nanocarriers and devices.¹² A large number of publications focus on the functionalization of polymeric devices such as catheters, grafts, cannulas, and sensors with NO donors or NO-donating moieties to reduce device-associated complications.^{9,10,12-15} Third, aqueous solutions, suspensions, and hydrogels containing NO donors are suited to be administrated via intranasal, intramuscular, intracameral routes, or topically for therapeutic purposes.¹⁶⁻¹⁹ They have also been employed as filling solutions for implants such as catheters,²⁰⁻²⁵ representing a unique method to release protective NO without modifying the polymers of medical implants.

Compared to NO delivery from NO tanks/generators and embedded solid NO donors, controlled release of NO from aqueous formulations is more challenging due to the low solubility, high reactivity, and toxicity of many NO donors. For example, sodium nitroprusside and N-diazeniumdiolates pose a risk of toxicity due to their degradation byproducts, including thiocyanate and nitrosamines.^{26,27} S-Nitroso-N-acetylpenicillamine, a commonly used synthetic S-nitrosothiol type NO donor, has a low aqueous solubility of only 2.1 mg/mL (<0.01 M) in water.²⁸ GSNO is a natural NO carrier and transporter circulating in the blood and occurring within the cytoplasm of cells.²⁹ Compared to other donors, GSNO is especially suitable for water-based drug formulations. As the S-nitrosated derivative of tripeptide glutathione, GSNO has an aqueous solubility of 0.075 M at low pH and >1 M at the physiological pH. In vivo studies did not reveal any toxicities of this natural NO donor when administrated in humans, dogs, and rats at appropriate doses^{30,31} There have been nearly 20 clinical trials using GSNO as a therapeutic drug, further confirming its safety.^{29,32} However, one long-standing and well-recognized challenge of NO delivery via GSNO solutions is the high GSNO reactivity that leads to “rapid and often unpredictable” rates of NO generation in medical applications.³² GSNO readily decomposes under heat (e.g., 37° C.) and light, and in the presence of catalysts/reactants such as metal ions, thiols, ascorbic acid, enzymes, and proteins.^{29,33} Its decomposition rate is also concentration dependent via a thyil radical-based autocatalysis mechanism.^{34,35}

A high NO donor concentration is needed in many preventative and therapeutic applications to release adequate NO from a limited volume of solution. Simply dissolving GSNO in an aqueous solution may not work due to its high reactivity at high concentrations at the physiological temperature.³⁵ The total NO release duration is usually too short to provide sustainable benefit and the high initial burst release causes cytotoxicity and multiple adverse effects.^{15,36} Indeed, the biological function of NO is often bidirectional and highly dependent on its concentration.³⁷ Consequently, the modulation of the GSNO reactivity for sustained release of NO with reduced initial burst is a key to the successful implementation of GSNO solutions in many medical applications. The encapsulation of GSNO in suspended nanocarriers based on poly(methyl)methacrylate, poly(lactic-co-glycolic acid), alginate, chitosan, and liposome has been reported with the aim of controlling the GSNO decomposition.^{19,38-40,12,41} However, these nanocarriers usually have low drug loadings and the actual NO release duration is only several hours to several days.

Thus, there is a need in the art for improved compositions and methods for regulating the decomposition of concentrated GSNO at physiological pH.

### SUMMARY

It has been demonstrated herein that GSNO at physiological pH forms inclusion complexes with various cyclodextrins (CDs), such as aCD. The rate of thermal decomposition, photodecomposition, and reactions with biological molecules of encapsulated GSNO (GSNO within an inclusion complex with one or more substituted or unsubstituted cyclodextrins) is significantly reduced compared to free GSNO. NO release in a more sustained and steady fashion has been obtained.

One aspect of the disclosure provides a composition comprising guest/host inclusion complexes, each complex comprising guest S-nitrosoglutathione (GSNO) and at least one substituted or unsubstituted cyclodextrin as the host. In some embodiments, the composition comprises two or more types of cyclodextrin. In some embodiments, the cyclodextrin is not S-nitrosylated. In some embodiments, the cyclodextrin is an acetylated, alkylated, hydroxyalkylated, methylated, hydroxyethylated, or hydroxypropylated cyclodextrin cyclodextrin. In some embodiments, the cyclodextrin is selected from the group consisting of of alpha cyclodextrin, gamma cyclodextrin, hydroxypropyl beta cyclodextrin, acetyl beta cyclodextrin, methyl alpha cyclodextrin, methyl beta cyclodextrin, 2-hydroxypropyl alpha cyclodextrin, 2-hydroxypropyl gamma cyclodextrin, 2-hydroxyethyl beta cyclodextrin, and 2,3,6-tri-o-methyl beta cyclodextrin. In some embodiments, the host cyclodextrin is not co-complexed with a polymer. In some embodiments, a molar ratio of GSNO to cyclodextrin is from about 1:10 to 10:1. In some embodiments, the composition is in a liquid solution dosage form. In some embodiments, the composition is in a liquid suspension dosage form. In some embodiments, the composition is in a gel dosage form. In some embodiments, the composition is in a dried dosage form.

Another aspect of the disclosure provides a method of delivering nitric oxide to a subject in need thereof comprising administering an effective amount of a composition as described herein to the subject.

Another aspect of the disclosure provides method of inhibiting microbial growth and/or clot formation on a surface, comprising contacting the surface with a composition as described herein. In some embodiments, the surface is on a catheter and the catheter is at least partially filled with a solution containing the composition.

Additional features and advantages of the present invention will be set forth in the description of disclosure that follows, and in part will be apparent from the description of may be learned by practice of the disclosure. The disclosure will be realized and attained by the compositions and methods particularly pointed out in the written description and claims hereof.

### BRIEF DESCRIPTION OF THE DRAWINGS

FIGS. 1A-E. (A) Decay curves of 0.1 M GSNO in 0.1 M PB at pH 7.4 and 37° C. without CD and with 0.1 M αCD, HP βCD, and γCD. (B) Decay curves of 0.1M GSNO in 0.1 M PB at pH 7.4 and 37° C. with 0, 0.05, 0.1, 0.15 M αCD and 0.15 M αCD+0.1 M γCD. (C) Decay curves of 0.05 M GSNO in 0.05 M PB at pH 7.4 and 37° C. with and without CDs. (D) Decay curves of 0.05 M GSNO in 0.05 M PB at pH 7.4 at room temperature with or without αCD. (E) The remaining GSNO after 48-h of storage of 0.05 M GSNO in 0.05 M PB solutions in the presence of 0.1 M CD derivatives in the dark at 37° C. All PB solutions contain 0.1 mM EDTA as a Cu²⁺ chelator.

FIG. 2. NO release from 3 mL of 0.1 M GSNO solutions with or without an equal mole of αCD at 37° C.

FIG. 3. Photos of agar hydrogels containing 0.1 M GSNO with and without an equal mole of αCD after different days of storage at 37° C.

FIGS. 4A-C. Decomposition of 0.1 M GSNO in 0.1 M PB with and without 0.1 M αCD in the presence of 10 mM L-ascorbic acid (A), 10 mM cysteine (B), and 20 mg/mL BSA (C) at pH 7.4 and 37° C.

FIGS. 5A-B. NO release from silicone catheters filled with 0.1 M GSNO (A) and 0.15 M GSNO (B) mixed with different concentrations of αCD and soaked in pH 7.4 PBSE at 37° C. The initial NO fluxes on day 0 are shown as numbers.

FIGS. 6A-B. Prevention of S. aureus biofilm growth on the extraluminal surface of silicone catheters by GSNO lock solutions. (A) Viable biofilm bacteria on the outer surface of silicone catheters filled with PB as the control, or PB containing 0.1 M αCD, 0.1 M GSNO, or 0.1M GSNO+0.1 M αCD after 3-day incubation with S. aureus at 37° C. (B) Comparison of the biofilm growth on the external catheter surface after incubation with the S. aureus culture at 37° C. for 1, 3, and 5 days. The catheter is filled with 0.1 M GSNO solution with and without 0.1 M αCD. *: p<0.05 compared to control. #: p<0.05 compared to αCD.

FIGS. 7A-B. Eradication of mature S. aureus biofilm on the extraluminal surface of silicone catheters by lock solutions containing 0.1 M GSNO with and without 0.1 M αCD. The viable bacteria were counted after 24-h (A) and 48-h (B) treatments. *: p<0.05 compared to control. #: p<0.05 compared to αCD.

FIGS. 8A-B. Prevention of intraluminal catheter infection by NO-releasing lock solutions containing 0.1 M GSNO with and without 0.1 M αCD compared to control solutions. Viable planktonic bacteria in lock solutions (A) and viable biofilm bacteria on the intraluminal surface (B) after 3-day incubation with S. aureus at 37° C. The dashed line at 400 CFU/cm² represents the detection limit of our method. *: p<0.05 compared to control, #: p<0.05 compared to αCD.

FIGS. 9A-D. Antibacterial effect of NO release solutions on multiple strains including MRSA (A), S. epidermidis (B), E. coli (C), and P. aeruginosa (D). Planktonic bacteria and biofilm bacteria on the silicone catheter surface were quantified after 3-day exposure to different NO release and control solutions. The dashed line represents the detection limit of the method. *p<0.05 compared to control, #p<0.05 compared to αCD.

FIG. 10. Viability of fibroblast L929 cells after 24-h incubation with silicone catheters locked with phosphate buffer, 0.1 M αCD solution, 0.1 M GSNO solutions with and without 0.1 M αCD.

FIGS. 11A-B. Photos of hydrogels on an LB-agar plate inoculated with S. aureus before (A) and after (B) 24-h incubation at 37° C.

### DETAILED DESCRIPTION

The formation of host-guest inclusion complexes is a common strategy in drug formulation to improve the physicochemical stability, solubility, dissolution rate, and bioavailability of drugs.⁴² Cyclodextrins may be used as host molecules for drug inclusion due to their low toxicity, wide availability, and low cost. The traditional notion is that with a hydrophobic interior and hydrophilic exterior, cyclodextrins form complexes with hydrophobic compounds. Thus, it was surprising when it was demonstrated herein that cyclodextrins form host-guest inclusion complexes with S-nitrosoglutathione (GSNO) which is extremely hydrophilic.

Cyclodextrins (CDs) are sugar molecules bound together in rings of various sizes. Specifically, the sugar units are called glucopyranosides glucose molecules that exist in the pyranose (six-membered) ring configuration. Naturally occurring a, P, and 7 CDs consist of 6, 7, and 8 glucopyranose units, respectively, and differ in their cavity size and solubility.⁴³ These parent CDs can be further chemically modified to provide various derivatives that possess more diversified physicochemical and biopharmaccutical properties.⁴⁴ CDs have a cone-shaped structure with a hydrophilic exterior and a lipophilic cavity and are, therefore, typically employed to encapsulate hydrophobic drugs via intermolecular forces.⁴³ Host-guest behavior can be manipulated by chemical modification of the hydroxyl groups. O-Methylation and acetylation are typical conversions. Propylene oxide gives hydroxypropylated derivatives. The primary alcohols can be tosylated. The degree of derivatization is adjustable, i.e. full methylation vs partial. In some embodiments, suitable CDs include an alpha, beta, or gamma CD or its derivative, e.g. an acetylated, alkylated, hydroxyalkylated, methylated, ethylated, hydroxyethylated, tosylated, propylated, or hydroxypropylated alpha, beta, or gamma CD. In some embodiments, suitable CDs include acetylated cyclodextrins, such as acetyl-beta-cyclodextrin with degree of substitution of 5-10 or acetyl-beta-cyclodextrin with degree of ˜7 (randomly substituted cyclodextrin).

In some embodiments, suitable substituted or unsubstituted CDs include, but are not limited to, alpha cyclodextrin, gamma cyclodextrin, hydroxypropyl beta cyclodextrin, acetyl beta cyclodextrin, methyl alpha cyclodextrin, methyl beta cyclodextrin, 2-hydroxypropyl alpha cyclodextrin, 2-hydroxypropyl gamma cyclodextrin, 2-hydroxyethyl beta cyclodextrin, and 2,3, 6-tri-o-methyl beta cyclodextrin.

GSNO is a small molecule nitric oxide donor that produces nitric oxide and exerts potent and broad-spectrum antibacterial activities via multiple nitrosylation and oxidation mechanisms toward enzymes, proteins, DNA, and lipids. GSNO alone or together with exogenous nitric oxide or with additional antibacterial or antimicrobial agents may reduce the growth of multidrug-resistant bacteria in both planktonic and biofilm form. GSNO is bactericidal against various strains of bacteria, including both Gram-positive and Gram-negative organisms, fungi, mycobacteria, parasites, and viruses.

GSNO has high reactivity during storage and use. The host-guest inclusion complexes with CD as described herein significantly modulate its reactivity and/or the NO release profiles. Without being bound by theory, the host molecule can form an inclusion complex with GSNO and protect it from decomposition/reaction. The complex can dramatically increase the GSNO stability at near neutral pH (pH 4-9) in the presence of an adequate concentration of buffer. The initial burst release of NO is suppressed, and the longevity of NO release is enhanced. As a result, the nitric oxide release is precisely tuned to meet various antimicrobial, antithrombotic, and anti-inflammatory needs.

CD modulates various reactions of GSNO. For example, it modulates thermal GSNO decomposition such as at physiological temperature. It modulates the photodecomposition of GSNO. It modulates GSNO decomposition in the presence of other reactants or catalysts such as metal ions, reducing agents (e.g., ascorbic acid and thiols), hemoglobin and hemoglobin derivatives, etc. It modulates GSNO's reactions with other molecules such as thiols, peptides, and proteins.

A higher concentration of host molecules leads to a higher degree of modulation of GSNO decomposition and reaction. However, these host molecules have limited solubility. In some embodiments, when combining two or more host molecules in one solution or hydrogel, the total quantity of host molecules is increased beyond the solubility of one host molecule (when host molecule 1 is saturated, host molecule 2 can still be added and dissolved). Thus, the modulation capability of GSNO stability is further enhanced compared to using one type of host molecule. In some embodiments, a composition/complex as described herein comprises two or more types of cyclodextrin, e.g. 3, 4, 5, 6, 7, 8 or more.

In some embodiments, if two CDs are incorporated, a molar ratio of the first CD to the second CD is from about 1:2 to 2:1. In some embodiments, a molar ratio of GSNO to total CD in the inclusion complex is from about 1:10 to about 10:1, e.g. from about 1:4 to about 4:1, e.g. from about 1:3 to 3:1 or from about 1:2 to 2:1.

CD host molecules and GSNO may be dissolved or suspended in a liquid or a gel or a polymer. Dry powders of the host molecule and GSNO may also be formulated. The inclusion complex may be dried before storage and use. CD host molecules may also be introduced in the synthesis process of GSNO. For example, when GSNO is synthesized from the nitrosation of glutathione, CD host molecules can be added together with reactants including glutathione and nitrosating agent so that the collected product will include complexes of GSNO and CD molecules. The host molecule and/or GSNO may be attached to other molecules or polymers (e.g., cyclodextrin attached to a polymer instead of pure cyclodextrin; GSNO attached to another drug instead of pure GSNO). Suitable polymers include, but are not limited to, fluoroethylpolymer, polytetrafluoroethylene (PTFE), polyetheretherketone (PEEK), ethylene tetrafluoroethylene (ETFE), paralene, a hydrophilic polymer, and the like. In some embodiments, the host cyclodextrin is not co-complexed with a polymer. In some embodiments, the cyclodextrin is not S-nitrosylated. In some embodiments, the GSNO and cyclodextrin incorporated within the inclusion complex are substantially pure. As used herein, the term “substantially pure” refers to a molecule having a purity of about 95% or greater as measured by HPLC as percent area.

In some embodiments, the GSNO and CD host molecules are dissolved or suspended in pure aqueous solvents. In some embodiments, the solvent is an organic solvent such as alkane diols and polymers of alkane diols. In some embodiments, the solvent is a mixture of water and one or more organic solvents.

As demonstrated herein, some cyclodextrins do not cause significant cell lysis while modulating GSNO decomposition, which is essential for many biomedical applications. They are useful for cell-contacting applications such as blood cell-contacting applications. One example application is lock solutions of intravascular catheters. These low-lysis cyclodextrins do not cause significant hemolysis.

Exemplary uses of the compositions described herein (e.g., solutions, suspensions, and hydrogels) include, but are not limited to: i) to prevent or inhibit infectious diseases, such as bacterial or viral infections including COVID, ii) to be used as lock solutions of IV catheters and inflation solutions of urinary catheters to prevent thrombosis and infections, iii) to be infused into insulin infusion cannula to increase its lifetime. The short longevity of insulin infusion cannulas has been a major challenge in diabetes management, iv) to promote wound healing, v) to be used as injectable drugs through intravenous, intramuscular, subcutaneous, and other routes to aid in the therapy of diseases such as stroke and cancer, vi) to be inhaled to treat pulmonary problems, and vii) to be used as hydrogel coatings on medical implants or devices to prevent complications such as infection and blood vessel stenosis.

Catheters such as central line catheters and urinary Foley catheters are always filled with lock solutions or inflation solutions. NO release from these solutions can reduce the infectious and/or thrombotic complications of catheters due to the potent antimicrobial and antiplatelet activities of NO. Since one solution may need to fill urinary catheters and central line catheters for days to multiple weeks, sustained NO release is necessitated. Second, NO reduces inflammation and infection of insulin infusion cannulas and can enhance the lifetime of such subcutaneously implanted cannulas. The tiny Teflon or stainless-steel cannulas cannot hold a significant amount of NO donors. Instead, concentrated GSNO may be infused into cannulas together with insulin to supply a larger amount of NO. The NO donor needs to undergo minimal degradation in the infusion pump for one week or more. Third, NO-releasing nasal sprays have proven to be highly effective in mitigating viral infections including COVID-19. The GSNO solution stabilized by CDs may be used for storage and releases NO for a prolonged period of time in the respiratory tract. GSNO-CD solutions with sustained NO delivery may also promote other therapeutic applications for diseases such as cancer, stroke, asthma, embolization, and cystic fibrosis.

Embodiments of the disclosure include methods of inhibiting microbial growth, e.g. bacterial or viral, on a medical device or implant by contacting the medical device or implant with a composition as described herein. Some embodiments include methods of inhibiting microbial growth on a catheter surface comprising at least partially filling the catheter with a solution containing a composition as described herein. In clinical applications, lock solutions are filled in intravascular catheters when not in use to primarily reduce clotting. As demonstrated herein, GSNO-CD solutions are highly effective in preventing and killing planktonic and biofilm bacteria.

As used herein, a “medical device” is any device intended for medical purposes. Exemplary types of a medical device include an instrument, apparatus, constructed element or composition, machine, implement, or similar or related article that can be utilized to diagnose, prevent, treat or manage a disease or other conditions. The medical devices provided herein may, depending on the device and the embodiment, be implanted within a subject, utilized to deliver a device to a subject or utilized externally on a subject. The medical devices provided herein are sterile and are subject to regulatory requirements relating to their sale and use. Representative examples of medical devices and implants include, for example, cardiovascular devices and implants such as implantable cardioverter defibrillators, pacemakers, stents, stent grafts, bypass grafts, catheters and heart valves; orthopedic implants (e.g., total or partial arthroplastic joints such as hip and knee prosthesis); spinal implants and hardware (spinal cages, screws, plates, pins, rods and artificial discs); a wide variety of medical tubes, cosmetic and/or aesthetic implants (e.g., breast implants, fillers); a wide variety of polymers, bone cements, bone fillers, scaffolds, and naturally occurring materials (e.g., heart valves, and grafts from other naturally occurring sources); intrauterine devices; orthopedic hardware (e.g., casts, braces, tensor bandages, external fixation devices, tensors, slings and supports) and internal hardware (e.g., K-wires, pins, screws, plates, and intramedullary devices (e.g., rods and nails)); cochlear implants; dental implants; medical polymers, a wide variety of neurological devices; artificial intraocular eye lenses, skin dressings (e.g., wound care dressings), and wearable devices. In certain embodiments, the medical devices may also include a plurality of biomedical devices that are used in clinical and biomedical research settings (e.g., PCR machines or any other research instruments).

The medical device may include a sensor, which is defined herein as a device that can be utilized to measure one or more different aspects of a body tissue (anatomy, physiology, metabolism, and/or function) and/or one or more aspects of the medical device. Representative examples of sensors suitable for use within the present invention include, for example, fluid pressure sensors, fluid volume sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, mechanical stress sensors and temperature sensors. Within certain embodiments the sensor can be a wireless sensor or, within other embodiments, a sensor connected to a wireless microprocessor. Within further embodiments, one or more (including all) of the sensors can have a Unique Sensor Identification number (“USI”) which specifically identifies the sensor and/or a Unique Device Identification number (“UDI”) with which the sensors can provide unique information of the associated medical device for tracking purposes of the medical device manufacturer, the health care system, and regulatory requirements.

Embodiments of the disclosure further include methods of preparing a composition as described herein, e.g. by following steps as set forth in the Example.

The compositions of the present disclosure may also contain other components such as, but not limited to, antioxidants, additives, adjuvants, buffers, tonicity agents, bioadhesive polymers, and preservatives. In any of the compositions of this disclosure, the mixtures are preferably formulated at about pH 5 to about pH 8. This pH range may be achieved by the addition of buffers to the composition. It should be appreciated that the compositions of the present disclosure may be buffered by any common buffer system such as phosphate, borate, acetate, citrate, carbonate and borate-polyol complexes, with the pH and osmolality adjusted in accordance with well-known techniques to proper physiological values.

An additive such as a sugar, a glycerol, and other sugar alcohols, can be included in the compositions of the present disclosure. Pharmaceutical additives can be added to increase the efficacy or potency of other ingredients in the composition. For example, a pharmaceutical additive can be added to a composition of the present disclosure to improve the stability of the bioactive agent, to adjust the osmolality of the composition, to adjust the viscosity of the composition, or for another reason, such as effecting drug delivery. Non-limiting examples of pharmaceutical additives of the present disclosure include sugars, such as, trehalose, mannose, D-galactose, and lactose.

In an embodiment, if a preservative is desired, the compositions may optionally be preserved with any well-known system such as benzyl alcohol with/without EDTA, benzalkonium chloride, chlorhexidine, Cosmocil® CQ, or Dowicil 200.

Further embodiments provide a method of delivering nitric oxide to a subject in need thereof comprising administering an effective amount of a composition as described herein to the subject. The compositions of the disclosure may be useful for the treatment of any disease or disorder that would benefit from the administration of nitric oxide. Exemplary diseases/disorders include, but are not limited to, viral or bacterial infection, cancer, stroke, asthma, embolization, cystic fibrosis, diabetes, inflammation, chronic obstructive pulmonary disease and other pulmonary diseases, and blood vessel stenosis.

A patient or subject to be treated by any of the compositions or methods of the present disclosure can mean either a human or a non-human animal including, but not limited to dogs, horses, cats, rabbits, gerbils, hamsters, rodents, birds, aquatic mammals, cattle, pigs, camelids, and other zoological animals.

In some embodiments, the active agent (e.g. inclusion complex) is administered to the subject in a therapeutically effective amount. By a “therapeutically effective amount” is meant a sufficient amount of active agent to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The active agent may be combined with pharmaceutically acceptable excipients. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

It is to be understood that this invention is not limited to any particular embodiment described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order, which is logically possible.

### EXAMPLE

### Summary

S-nitrosoglutathione (GSNO) is a non-toxic nitric oxide (NO)-donating compound that occurs naturally in the human body. The use of GSNO to deliver exogenous NO for therapeutic and protective applications is limited by the high lability of dissolved GSNO in aqueous formulations. Here, we report a host-guest chemistry-based strategy to modulate the GSNO reactivity and NO release kinetics for the design of anti-infective catheters and hydrogels. Cyclodextrins (CDs) are host molecules that are typically used to encapsulate hydrophobic guest molecules into their hydrophobic cavities. However, we found that CDs form inclusion complexes with GSNO, an extremely hydrophilic molecule with a solubility of over 1 M at physiological pH. More interestingly, the host-guest complexation reduces the decomposition reactivity of GSNO in the order of αCD>γCD>hydroxypropyl βCD. The lifetime of 0.1 M GSNO is increased to up to 15 days in the presence of CDs at 37° C., which is more than twice the lifetime of free GSNO. Quantum chemistry calculations indicate that GSNO in uCD undergoes a conformational change that significantly reduces the S—NO bond distance and increases its stability. The calculated S—NO bond dissociation enthalpies of free and complexed GSNO well agree with the experimentally observed GSNO decomposition kinetics. The NO release from GSNO-CD solutions, compared to GSNO solutions, has suppressed initial bursts and extended durations, enhancing the safety and efficacy of NO-based therapies and device protections. In an example application as an anti-infective lock solution for intravascular catheters, the GSNO-αCD solution exhibits potent antibacterial activities for both planktonic and biofilm bacteria, both intraluminal and extraluminal environments, both prevention and treatment of infections, and against multiple bacterial strains including a multidrug-resistant strain. In addition to solutions, the inclusion complexation also enables the preparation of GSNO hydrogels with enhanced stability and improved antibacterial efficacy. Since methods to suppress and control the GSNO decomposition rate are rare, this supramolecular strategy provides new opportunities for the formulation and application of this natural NO donor.

### Materials and Methods

### Chemicals and Reagents

Sodium phosphate dibasic (Na₂HPO₄), sodium hydroxide (NaOH), ethylenediaminetetraacetic acid disodium salt dihydrate (EDTA·2Na), L-glutathione reduced (GSH), cysteine, L-ascorbic acid, bovine serum albumin (BSA), and sodium nitrite were purchased from Millipore Sigma. Luria-Bertani (LB) broth powder, agar, αCD, Dulbecco's modified Eagle's medium (DMEM), fetal bovine serum (FBS), and penicillin/streptomycin were purchased from Thermo Fisher Scientific. Gamma CD and 2-hydroxypropyl βCD (HP βCD) were purchased from Tokyo Chemical Industry and Cayman Chemical Company, respectively. Other CD derivatives were purchased from Cyclolab Ltd. Bacterial strains including S. aureus (25923), methicillin-resistant S. aureus (MRSA, BAA-2312), S. epidermidis (12228), E. coli (53496), and P. aeruginosa (baa-744) as well as murine fibroblast cell line (L929) were purchased from the American Type Culture Collection (ATCC). The CellTiter 96® AQueous One Solution Cell Proliferation Assay (MTS) kit was obtained from Promega Corporation.

### GSNO Synthesis and Preparation of NO Release Solutions

GSNO was synthesized by nitrosation of reduced L-glutathione in acidified nitrite as previously reported.⁵² After collecting GSNO precipitates and washing them using cold water and acetone, GSNO powder was vacuum dried for 4 h and stored at −20° C. GSNO powders were dissolved in phosphate buffer (PB) containing the same mole of Na₂HPO₄ before the pH adjustment. For example, the formal concentration of Na₂HPO₄ is 0.1 M when the GSNO concentration is 0.1 M. One hundred μM EDTA was always added to each solution before the solution pH was finally adjusted to 7.4 via NaOH. EDTA is a chelating agent to mask Cu²⁺, a S-nitrosothiol catalyst that may exist in solvents and chemicals as an impurity.⁵³ For solutions with reactive agents, BSA, ascorbic acid, or cysteine was added before the pH adjustment. To prepare GSNO-CD solutions, appropriate amounts of CD powders were dissolved into the GSNO solutions prior to the pH adjustment.

### Measurement of GSNO Decomposition Via UV-Vis Absorption Spectroscopy

Aqueous GSNO solutions were contained in 1.5 mL disposable polystyrene cuvettes. The cuvette was caped and further sealed with parafilm. The concentrations of GSNO during storage at 37° C. or 25° C. in the dark were monitored by a UV-vis spectrophotometer (Go Direct® Fluorescence/UV-VIS Spectrophotometer) at a wavelength of 545 nm. A 4 W LED white light was used as the light source for the photodecomposition experiment at room temperature. The distance between the light source and the cuvettes was approximately 10 cm. All GSNO decomposition experiments were triplicated, and the GSNO concentration was expressed as mean±standard deviation.

### Measurement of NO Release

The NO release was quantified by an ECO PHYSICS NO analyzer (nCLD 66). Two L of castor oil was added to NO release solutions as an antifoam agent when 3 mL GSNO or GSNO-CD solutions were tested in an amber glass cell at 37° C. Humidified air was vacuumed into the solution to carry NO continuously into the chemiluminescence detection chamber at a flow rate of 100 cm³/min. The solutions were stored in sealed amber glass vials between measurements at 37° C. To monitor the NO release from catheters, solutions were filled into 2.5 cm-long medical-grade silicone tubes (HelixMark® 60-011-07, 1.58 mm ID, 2.41 mm OD) that were scaled on both ends with plastic rods. The filled tubes were placed into the amber glass cell containing 5 mL of 0.01 M PBS with 0.1 mM EDTA (PBSE) at 37° C. N₂ was used as the carrier gas at a flow rate of 100 cm³/min to introduce NO into the analyzer. Filled tubes were stored in 10 mL PBSE at 37° C. in the dark between measurements. The soaking PBSE solution was refreshed after each measurement.

### Preparation of Agar Hydrogel Containing GSNO

Two grams of agar were dissolved in 100 mL of 0.1 M phosphate buffer at pH 7.4 at 80° C. for 3 h with stirring. GSNO solutions at 0.2 M with or without 0.2 M αCD in 0.1 M phosphate buffer at pH 7.4 were prepared separately. Then, 0.5 mL GSNO solution and 0.5 mL dissolved agar were mixed and put into an ice bath to facilitate gelation.

### In Vitro Antibacterial Experiments

Both biofilm and planktonic bacteria were quantified to evaluate the antibacterial activities of the proposed method. LB medium (25 mg/mL) was used to culture all bacterial strains overnight before performing the antibacterial tests. Segments of medical-grade silicone tubes were all autoclaved. Lock solutions were freshly prepared using the sterilized buffer and filtered through a 0.22 m PES syringe filter. The biofilms on the silicone surface were dip-rinsed 5 times in PBS and detached by 1-min vertexing, 1-min sonication (20% power of a 150 W probe sonifier, Branson Ultrasonics), and another 1-min vertexing for plate counting. When quantifying the planktonic bacteria, the suspension was homogenized by 1-min vertexing. Bacterial suspensions were serially diluted using sterile PBS prior to inoculating the 1.5 wt % agar LB plates. After plate incubation at 37° C. overnight, colony-forming units (CFU) of bacteria were counted and converted to CFU/cm² for biofilm quantification and CFU/mL for planktonic bacteria quantification. An acceptable count of CFU is 20-200 per plate. In our case, the limit of detection (LOD) is 400 CFU/cm² for biofilm quantification and 200 CFU/mL for quantification of planktonic bacteria. All bacterial tests were triplicated. Data were reported as mean±standard deviation. The statistical significance between groups was determined using a student's t-test.

### Extraluminal Bacteria Assessment

The overnight S. aureus culture was diluted by LB (0.25 mg/mL) medium to approximately 10³ CFU/mL. Silicone rubber tubing was cut into 2.7 cm segments and one end was sealed with RTV silicone rubber glue. The silicone segment was filled with a lock solution and then transferred to a 15-mL centrifuge tube with 1.8 mL bacterial culture. The top end of the catheter segment was left open and above the liquid culture. Centrifuge tubes were capped loosely and incubated statically at 37° C. in an incubator. The catheter segments were dip-rinsed 5 times in sterile PBS to remove the loosely attached bacteria before the broth was refreshed once daily. At the end of the experiment, the lock solution was completely extracted and discarded. The empty silicone tube was thoroughly rinsed and transferred to 5 mL PBS to perform the biofilm evaluation experiment detailed above. Glutathione (0.25 mg/mL) is present in the LB medium to mimic the thiol environment of blood. To simulate the lock solution therapy for bacteria eradication, silicone segments were capped with plastic plugs at both ends. Bacterial biofilms were formed on the exterior surface after incubating the sealed segment in S. aureus culture for 48 h at 37° C. Then, the catheter lumen was filled with different lock solutions. The infected silicone tubes were transferred to 15-mL centrifuge tubes with PBS and incubated for 24 or 48 h at 37° C. The viable bacteria on the outer surface were counted following the treatment.

### Intraluminal Bacteria Assessment

The 0.1 M αCD, 0.1 M GSNO, and 0.1 M GSNO+0.1 M αCD solutions were prepared in LB broth buffered with 0.1 M Na₂HPO₄ and adjusted to pH 7.4. Then, the overnight bacterial cultures were 100-fold diluted by the buffered LB broth, αCD, GSNO, or GSNO+αCD solutions. Silicone tubing was cut into 2 cm segments and then cut longitudinally into two halves to fully expose all surfaces, mimicking the interaction between the inner silicone surface and the lock solution. The silicone pieces were soaked into the prepared solutions with bacteria and statically incubated for 3 days at 37° C. Viable biofilm and planktonic cells were evaluated by plate counting. To simulate the lock therapy for intraluminal bacterial biofilm, sterile silicone pieces were exposed to S. aureus for 48 h at 37° C. to grow biofilm. Then, these pieces were treated with different lock solutions for another 24 h followed by the biofilm evaluation process described above.

### Agar Diffusion Assay for the Hydrogel Containing GSNO

The overnight S. aureus culture was diluted to a density of 0.1 OD₆₀₀ in PBS. Then, the diluted bacterial inoculum was uniformly spread on an LB-agar plate using a sterile cotton swab. The plate was marked as four separate portions. Forty μL of warm liquid agar with only buffer, GNSO, αCD, or GSNO-αCD (see section 2.5) was added onto a portion of the inoculated plate twice. The agar solution was gelated almost instantly due to the temperature decrease to form a small circular hydrogel area. The bacterial growth was visually examined after 24-h incubation at 37° C.

### Cytotoxicity Test

L929 murine fibroblast cells were suspended in complete media (DMEM with 1% penicillin/streptomycin and 10% FBS) before being seeded in a 24-well plate at a concentration of 5×10⁵ cells per well. After 24 h of incubation, the attached cells in each well were incubated in fresh media containing a sterilized 1-cm silicone tube (HelixMark® 60-011-07) filled with a lock solution with buffer, GSNO, or GSNO-αCD and with both ends sealed. MTS assays were performed following the manufacturer's procedure after an additional 24-h incubation. Absorbance at 490 nm was measured by a Cytation 3 imaging plate reader (Biotek).

### Results and Discussion

### Effect of CDs on the Thermal Decomposition of GSNO in Aqueous Solutions and Hydrogels

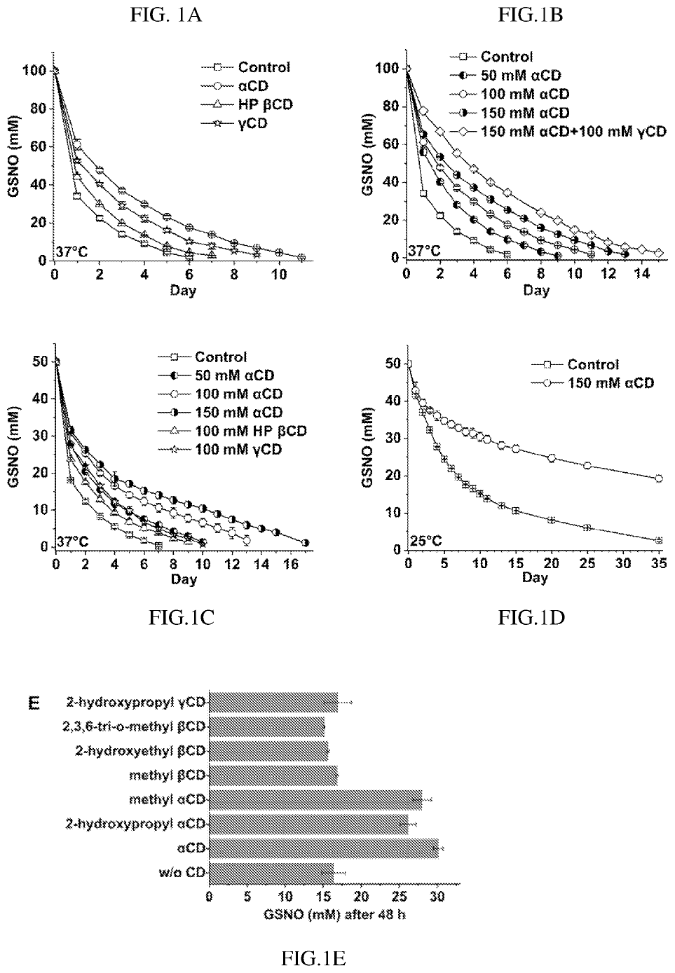

GSNO decomposes to liberate NO at the physiological temperature via thermal cleavage of its S—NO bond. Using an optimized buffer system according to our previous work,⁵⁸ 0.1 M GSNO lasts 6 days in 0.1 M PB at pH 7.4 and 37° C. (FIG. 1A, black line). However, 66% of GSNO degrades within the first 24 h, suggesting an initial burst release of NO. When the GSNO solution contains 0.1 M CD, the GSNO stability is enhanced in the order of αCD>γCD>HP βCD (hydroxypropyl βCD). The solubility of parent βCD in water is only 16.5 mM, which is inadequate to complex a high concentration of GSNO. So, we chose a commonly used derivative, HP βCD as a more soluble substitute. An equal mole of αCD increases the GSNO lifetime to 11 days, which is nearly twice the lifetime of GSNO only. The GSNO decay in the first 24 h also decreases to only 39%, suggesting a much lower initial decomposition reactivity in the presence of the hosting αCD. As shown in FIG. 1B, the stabilization effect is dependent on the molar ratio of the host and guest molecules. When the αCD concentration is increased to its maximum solubility (0.15 M), the GSNO decomposition rate is further reduced as the GSNO-CD binding equilibrium further shifts to the right. To overcome the solubility limit, multiple CDs may be used at high concentrations together to inhibit the GSNO decomposition. The GSNO lifetime is increased to 15 days with a first-day decay of only 22% in the presence of 0.15 M αCD and 0.1 M γCD (FIG. 1B). The lifetime and first-day decay are improved by 2.5 and 3 times, respectively. Compared to 0.15 M αCD only, the addition of 0.1 M γCD presumably allows for inclusion complexation of more GSNO and leads to further enhanced stability of GSNO. When the concentration of GSNO is reduced to 0.05 M, similar trends of stabilization were observed in the presence of different CDs and different concentrations of αCD (FIG. 1C). The GSNO lifetime is 17 days when 0.05 M GSNO and 0.15 M αCD are dissolved in 0.05 M PB at pH 7.4 at 37° C., as opposed to only 7 days without CDs. The stabilization effect is also pronounced at room temperature. As shown in FIG. 1D, the half-life of GSNO is ˜20 days in the presence of 0.15 M αCD, which is 4 times that of only GSNO.

We further examined six other CD derivatives in a 2-day experiment and found that parent αCD is still the best (FIG. 1E). If we group the derivatives based on their parent CDs, the stabilization effect also follows the order of αCD>γCD>βCD. Generally, βCD and its derivatives are the most commonly used and successful complexing agents in pharmaceutical formulations as their cavities match the size of many drugs.⁴⁵ Our results indicate that αCD is most effective in modulating the GSNO reactivity. αCD derivatives, including methyl αCD and 2-hydroxypropyl αCD, also show a significant stabilization effect, but these derivatives are more expensive than the parent αCD and therefore not further studied in this work.

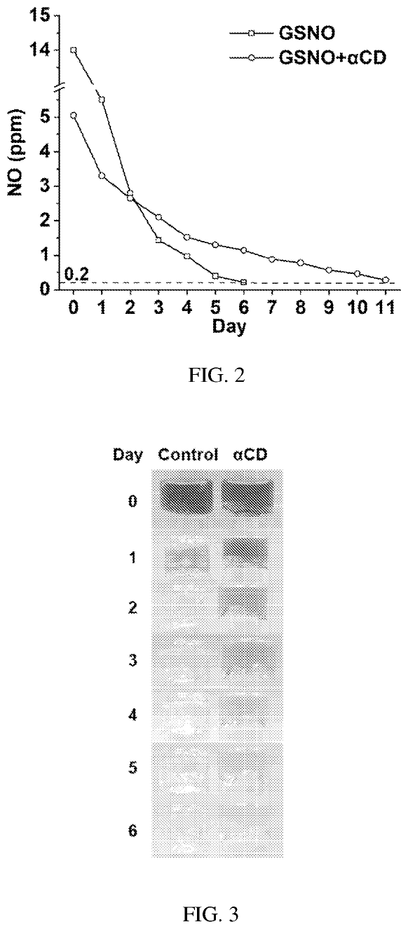

The decomposition of each GSNO molecule liberates one NO. Parent CDs, alkylated CDs, and hydroxyalkylated CDs examined in this study are not expected to react with NO and consume NO. Therefore, the decay kinetics of GSNO based on absorption spectroscopy should agree with the NO generation kinetics. To confirm this, NO release from 3 mL of 0.1 M GSNO solution (3×10⁻⁴ moles GSNO) with and without αCD is tested using a chemiluminescence-based NO analyzer. As shown in FIG. 2, GSNO and GSNO-αCD solutions release NO for 6 and 11 days, respectively, which is completely consistent with the absorbance-based GSNO decay data. The cumulative NO release is calculated to be 2.48×10-⁴ and 2.36×10-⁴ moles, respectively, confirming that the presence of αCD does not consume NO.

NO-releasing hydrogels have been shown to promote wound healing in topical applications,⁵⁹ enhance tumor immunotherapy as injectables,⁶⁰ and protect medical implants as coatings.⁸ Therefore, we studied the effect of αCD on the stability of GSNO in hydrogel using agar hydrogel as an example. As shown in FIG. 3, the reddish color of GSNO in 1% agar hydrogel fades in less than 2 days at 37° C., whereas the color lasts more than 6 days in the presence of an equal mole of αCD, suggesting that CD is a versatile host molecule to enhance GSNO stability in various water-based media. GSNO solutions have also been used in the preparation of NO-releasing water-in-oil emulsions¹⁹ and liposomes⁴⁰, where the addition of CDs is also expected to modulate the GSNO decomposition and NO release.

### Stabilization Effect of αCD on GSNO in the Presence of Light and Biologically Relevant Reactants

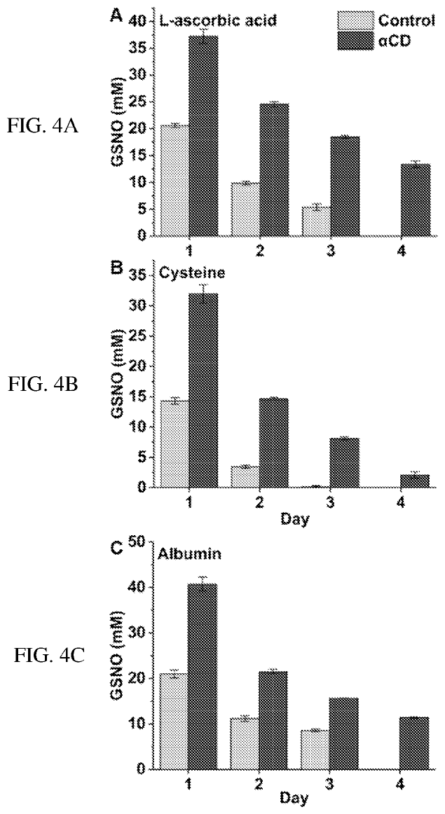

When GSNO solutions are mixed or in contact with biological fluids, the GSNO decomposition and NO release are expected to be faster due to the presence of enzymes such as GSNO reductase and 7-glutamyl transpeptidase, reactants such as ascorbic acid, thiols, metal ions, and proteins, as well as NO scavengers like hemoglobin.^{29,33} Therefore, we further studied the effect of CD complexation on these reactions. Cysteine and ascorbate cause reductive cleavage of the S—N bond in GSNO and other S-nitrosothiols. As shown in FIGS. 4A and 4B, the addition of 10 mM cysteine and L-ascorbic acid reduces the lifetime of 0.1 M GSNO to 2-3 days in 0.1 M PB at pH 7.4, much shorter than the 6-day lifetime without these reductive compounds. However, the addition of 0.1 M αCD remarkably reduces the decomposition rate in the 4-day experiments. Although the actual cysteine and ascorbate concentration in vivo is lower than the used 10 mM, multiple reductive reactants coexist in real biofluids such as blood and may accelerate the GSNO decomposition together. The purpose of this experiment is to clearly show that the host-guest interaction modulates the reaction of GSNO in the presence of biologically relevant reactants instead of implying specific reaction rates in real biological environments. S-transnitrosylation of proteins, including albumin, by GSNO is one of the in vivo generation mechanisms of S-nitrosated proteins, such as S-nitrosoalbumin, the most abundant circulating NO carrier in the blood.⁶³ To explore whether CDs could regulate the reaction of GSNO with proteins, BSA was added to GSNO solutions with and without αCD. As can be seen from FIG. 4C, 41% GSNO is left after 24 h of storage at 37° C. in the presence of 0.1 M αCD, whereas only 21% is left in the control group. Photolytic decomposition of GSNO and other S-nitrosothiols is another widely studied reaction for NO generation. The presence of αCD leads to a nearly 2-fold decrease in the decomposition rate of GSNO in a 4-h experiment under LED white light. Collectively, the inclusion complexation of GSNO by CD is an effective method to reduce GSNO reactivity in various reactions and conditions.

No Release and Antibacterial Properties of Catheters Filled with GSNO-CD Solutions

The GSNO-CD solutions with controlled and sustained release of NO have a variety of potential applications. First, catheters such as central line catheters and urinary Foley catheters are always filled with lock solutions or inflation solutions. NO release from these solutions can reduce the infectious and/or thrombotic complications of catheters due to the potent antimicrobial and antiplatelet activities of NO.²⁰⁻²⁵ Since one solution may need to fill urinary catheters and central line catheters for days to multiple weeks, sustained NO release is necessitated. Second, we and other groups found that NO reduces inflammation and infection of insulin infusion cannulas and holds promise in enhancing the lifetime of such subcutaneously implanted cannulas.^{64,65} The tiny Teflon or stainless-steel cannulas cannot hold a significant amount of NO donors. Instead, concentrated GSNO may be infused into cannulas together with insulin to supply a larger amount of NO. The NO donor needs to undergo minimal degradation in the infusion pump for one week or more. Third, NO-releasing nasal sprays have proven to be highly effective in mitigating viral infections including COVID-^{19.66} The GSNO solution stabilized by CDs may be a formulation that is amenable for storage and releases NO for a prolonged period of time in the respiratory tract. GSNO-CD solutions with sustained NO delivery may also promote other therapeutic applications for diseases such as cancer, stroke, asthma, embolization, and cystic fibrosis.^{18,29,32,67}

Here, we use lock solutions of central line catheters as an example application of GSNO-CD solutions and focus on the antibacterial activities of NO to prevent and treat infections. More than 5 million central venous catheters are inserted annually in the US to provide venous access for kidney dialysis, anti-cancer drug infusion, total parenteral nutrition, long-term antibiotic treatment, and repeated drawing of blood samples. Approximately 30,000 episodes of central line-associated bloodstream infections occur in USA acute care hospitals annually.⁶⁸ There are also ˜30,000 bloodstream infections in outpatient hemodialysis facilities annually in the US, 76.5% of which are related to hemodialysis catheters.⁶⁹ Compared to coating or doping bactericidal agents onto or into polymeric catheters, locking catheters with antimicrobial solutions between uses is a replenishable strategy without limitations in drug loading. Direct use of commercial catheters without any device modification is another advantage of the lock solution method. Antibiotics and ethanol are currently used in clinical applications as anti-infective lock solutions.⁴⁹ Other drugs like taurolidine are being developed for catheter locking applications.⁷⁰ One obvious drawback of these locking solutions is that the large organic drugs cannot pass through the wall of a catheter and therefore cannot protect the exterior surface of the catheter at all although bacterial contamination may occur extraluminally due to open surgical site contamination, exit-site infection or bacteremia caused by other sources.⁷¹ Due to the high diffusivity of NO through the polymer materials,⁷² the NO release solution can fully protect the entire catheter (both intraluminal and extraluminal surfaces) from bacterial colonization.

No Release of Silicone Rubber Tubes Filled with GSNo-αCD Solutions

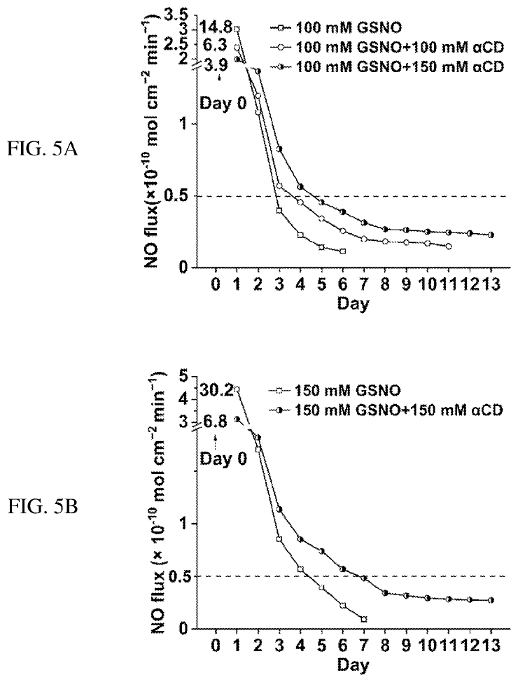

The NO generation from the solution itself has been tested (FIG. 2), which is indicative of the NO generation within the catheter lumen from the GSNO-CD lock. To evaluate the NO release from the exterior catheter surface, we filled medical-grade silicone rubber tubes with GSNO or GSNO-αCD solutions and sealed both ends. As shown in FIG. 5A, NO indeed penetrates through the polymeric catheter wall to reach the outer catheter surfaces and be detected by the NO analyzer. The 0.1 M GSNO solution and 0.1 M GSNO+0.1 M αCD solution release NO over 0.1×10⁻¹⁰ mol cm⁻² min⁻¹ for 6 days and 11 days, respectively, from the catheter exterior. αCD suppresses the initial release of NO from 14.8×10⁻¹⁰ mol cm⁻² min⁻¹ to 6.3×10⁻¹⁰ mol cm⁻² min⁻¹ on day 0. Increasing αCD to 0.15 M further extends the NO release duration and reduces the initial release to 3.9×10⁻¹⁰ mol cm⁻² min⁻¹, which is comparable to the 0.5-4×10⁻¹⁰ mol cm⁻² min⁻¹ flux of NO released from the healthy endothelium in the human body.⁷³ The extraluminal NO release from catheters can be further adjusted by changing the concentration of GSNO. For example, a one-week release of NO above a flux of 0.5×10⁻¹⁰ mol cm⁻² min⁻¹ is obtained by using 0.15 M GSNO+0.15 M αCD (FIG. 5B). In previous studies using nitrite or S-nitrosothiol-based solutions/suspensions as filling solutions of catheters, only less than 2 days of NO release with initial bursts was reported, and the antibacterial efficacy was only assessed with <24 h of locking using NO donor solutions.^{20-23,25} Given that central line catheters need to be locked for 2 or 3 days to several weeks in hemodialysis and chemotherapy and the GSNO reactivity is higher in the real biological environment containing hemoglobin and other reactive species,^{29,33,63,74} more steady and sustained NO release from the GSNO-CD solutions is beneficial. Different catheter materials and wall thicknesses may lead to different NO fluxes,⁷² but the composition of the lock solution can be adjusted to attain desirable NO levels from the outer catheter surfaces.

### Efficacy of the GSNO-αCD Solution in Preventing and Treating Extraluminal Catheter Infections

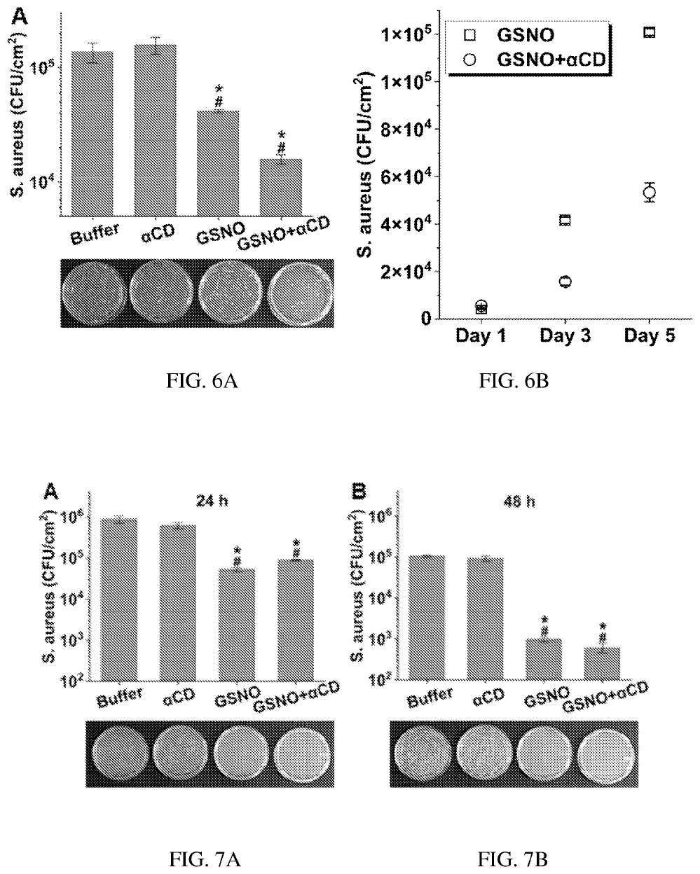

Intravascular catheters are prone to microbial biofilm colonization on intraluminal and extraluminal surfaces.⁷¹ Prevention and treatment of extraluminal biofilms by traditional antimicrobial lock solutions is hardly possible because the drug resides within the catheter lumen and cannot penetrate the catheter wall. Herein, we assess the effectiveness of GSNO-αCD lock solution against extraluminal biofilm formation using S. aureus, the most causative microorganism in intravascular catheter-associated infections,⁷⁵ as an example. The S. aureus biofilm on the outer surface of silicone rubber catheters filled with different NO release and control solutions is quantified using the plate counting method. As shown in FIG. 6A, the GSNO only and GSNO-αCD solutions reduce bacterial biofilm by 70% and 88%, respectively, compared to the control solutions using phosphate buffer and αCD in 3-day experiments. Because of the sustained NO release of GSNO when complexed with αCD, the antibacterial effect is also more sustainable. As shown in FIG. 6B, although the GSNO-αCD group shows slightly more bacteria than the GSNO group on day 1 due to the lower initial flux, its biofilm bacteria become significantly less on days 3 and 5 as the NO flux from GSNO-CD surpasses that from GSNO only.

Eradicating established biofilms on the catheter surface is another clinical need because removing a contaminated catheter is not always a first-line option. Current guidelines recommend sterilizing a previously infected catheter with a highly concentrated antibiotic (100-1,000 times planktonic minimum inhibitory concentration) for 24 to 48 h to eliminate biofilms.⁴⁹ Again, this method is effective primarily for bacterial biofilms on the intraluminal surface rather than the extraluminal surface. NO is a unique therapeutic agent against mature biofilms because it can easily penetrate biofilms to disperse and/or kill bacteria.⁷⁶ Therefore, the NO release from GSNO-αCD solutions is used to remove S. aureus biofilms developed on the outer catheter surface. Silicone rubber tube segments with plugged ends were exposed to S. aureus culture for 48 h to grow biofilms on the outer surface. Then, these tubes were filled with different lock solutions for 24 h with both ends sealed before the bacterial biofilm was quantified. As shown in FIG. 7A, both NO release solutions reduce the number of viable S. aureus on the outer surface by at least 90% compared to the control catheters treated with phosphate buffer or αCD. The slightly high efficacy of the GSNO solution without αCD after 24 h agrees with its higher NO flux in the first day. After 48 h, the GSNO-αCD solution becomes more effective due to its higher NO flux on day 2 (FIG. 7B). More than 99% of mature S. aureus biofilm on the outer surface is removed after the 48-h treatment using the GSNO-CD solution, representing a unique lock therapy for the eradication of extraluminal bacterial biofilms. The 1-log reduction of biofilm bacteria in control groups from 24 to 48 h might be caused by the detachment of some mature bacteria from the catheter surface.

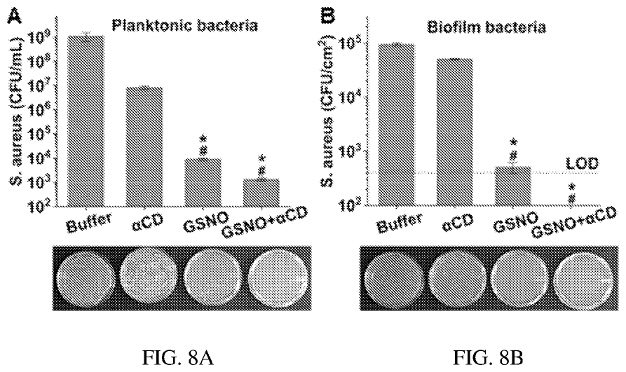

Efficacy of the GSNO-αCD solution in preventing and treating the intraluminal catheter infections Bacteria also enter the catheter lumen from hub region contamination and colonize the luminal surface to form biofilms.⁷⁷ Unlike exterior surfaces that are only exposed to NO diffused through the catheter wall, the lumen is filled with the lock solution. An antibacterial lock solution should kill planktonic bacteria in the solution and inhibit biofilm development on the intraluminal surface. Therefore, we assessed the growth of both planktonic and biofilm bacteria in the presence of different lock solutions in 3-day experiments. As shown in FIG. 8, the growth of planktonic S. aureus is reduced by 5 and 6 orders of magnitude by the GSNO and GSNO-αCD solution, respectively, compared to the phosphate buffer. The viable S. aureus in the biofilm on silicone rubber is undetectable when the silicone rubber is exposed to the GSNO-αCD solution. The bacterial biofilm is reduced by 99.2% in the GSNO only group but more than the GSNO-αCD group, which again agrees with the higher NO flux generated by the GSNO-αCD inclusion complex after the first day. If the S. aureus biofilm is already established on the silicone rubber surface, the use of GSNO-αCD solution completely eradicates the biofilm after one day of lock therapy.

### Broad-Spectrum Activities of NO Against Multiple Bacterial Strains

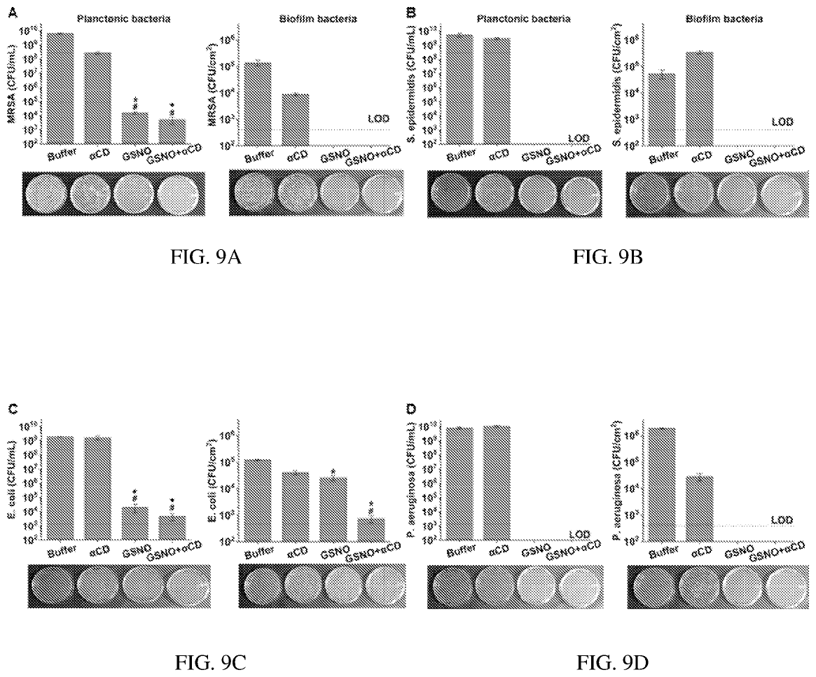

NO is known to exert broad-spectrum antibacterial activities based on multiple nitrosylation and oxidation mechanisms toward enzymes, proteins, DNA, and lipids.⁷⁸ To confirm the efficacy of GSNO and GSNO-αCD solution against other bacteria, we tested two more gram-positive strains (MRSA and S. epidermidis) and two gram-negative strains (E. coli and P. aeruginosa). FIG. 9 shows the viable planktonic bacteria after 3 days of incubation with different NO release and control solutions. The GSNO-αCD solution results in at least 6-log reductions of all bacterial strains, confirming the highly broad-spectrum antibacterial activities of NO. Similarly, the formation of bacterial biofilm is also inhibited by NO release solutions to the level of being undetectable under our experimental conditions except E. coli. The GSNO solution only reduces E. coli biofilm by 79% while the GSNO-αCD solution leads to 99% reduction. The reason of the low efficacy of the GSNO solution in preventing E. coli biofilm formation on the silicone surface is not fully understood, but it may be related to the reaction of NO donors with indole generated by E. coli as demonstrated in our recent publication.⁷⁹ This reaction may consume NO donors and compromise the NO release. The much better efficacy of GSNO-αCD solution is an interesting observation, suggesting that αCD probably suppresses such reactions as it does for other GSNO reactions and preserves the NO for bacterial inhibition.

### Cytocompatibility of the NO-Releasing Catheter

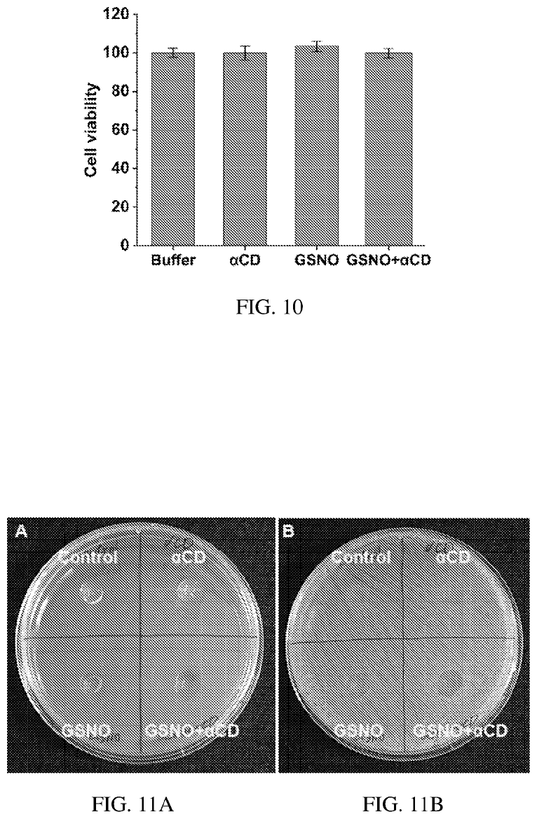

MTS assays were performed to evaluate the possible cytotoxicity of the NO-releasing catheter filled with the GSNO or GSNO-αCD solution as a lock solution. As shown in FIG. 10, there is no difference in the cell viability between silicone rubber tubes filled with NO-releasing solutions, phosphate buffer, and αCD, indicating that the NO release induces no cytotoxicity to mouse fibroblast L929 cells.

### Antibacterial Property of the GSNO-αCD Hydrogel

We also tested the antibacterial property of the NO-releasing agar hydrogel using the agar diffusion method. As shown in FIG. 11, the agar hydrogel containing GSNO-αCD grows fewest S. aureus (less white) and the GSNO hydrogel also grows less bacteria compared to the hydrogel with buffer or αCD. This is consistent with the high antibacterial activity of GSNO-αCD solution.

### CONCLUSIONS

We demonstrated that GSNO at near-neutral pH forms complexes with CDs and becomes chemically more stable. αCD is most effective in stabilizing GSNO due to the highest bond dissociation energy of S—NO after the conformational change of GSNO in the CD cavity. The GSNO-αCD solution releases NO with a significantly reduced initial burst and an extended duration. The sustained NO release from the GSNO-CD solution and hydrogel enables highly effective prevention and treatment of bacterial infections. In addition to thermal decomposition, the reactivity of GSNO in the presence of biological catalysts and light is also regulated by the host-guest complexation. As there are many more CD derivatives and other macrocyclic molecules available, more NO release profiles may be obtained from host-encapsulated GSNO. Our new GSNO formulations are also expected to advance more applications of NO such as thrombosis inhibition, cancer therapy, virus eradication, and wound healing.

### REFERENCES

(1) Loscalzo, J.; Welch, G. Nitric Oxide and Its Role in the Cardiovascular System. Prog. Cardiovasc. Dis. 1995, 38 (2), 87-104.
(2) Sharma, J. N.; Al-Omran, A.; Parvathy, S. S. Role of Nitric Oxide in Inflammatory Diseases. Inflammopharmacology 2007, 15 (6), 252-259.
(3) Kiss, J. P. Role of Nitric Oxide in the Regulation of Monoaminergic Neurotransmission. Brain Res. Bull. 2000, 52 (6), 459-466.
(4) Bogdan, C. Nitric Oxide and the Immune Response. Nat. Immunol. 2001, 2 (10), 907-916.
(5) Luo, J.; Chen, A. F. Nitric Oxide: A Newly Discovered Function on Wound Healing. Acta Pharmacol. Sin. 2005, 26 (3), 259-264.
(6) Seabra, A. B. Nitric Oxide Donors: Biomedical Applications and Perspectives. Academic Press 2017.
(7) Carpenter, A. W.; Schoenfisch, M. H. Nitric Oxide Release: Part II. Therapeutic Applications. Chem. Soc. Rev. 2012, 41 (10), 3742.
(8) Chen, Y.; Gao, P.; Huang, L.; Tan, X.; Zhou, N.; Yang, T.; Qiu, H.; Dai, X.; Michael, S.; Tu, Q.; Huang, N.; Guo, Z.; Zhou, J.; Yang, Z.; Wu, H. A Tough Nitric Oxide-Eluting Hydrogel Coating Suppresses Neointimal Hyperplasia on Vascular Stent. Nat. Commun. 2021, 12 (1), 7079.
(9) Rong, F.; Tang, Y.; Wang, T.; Feng, T.; Song, J.; Li, P.; Huang, W. Nitric Oxide-Releasing Polymeric Materials for Antimicrobial Applications: A Review. Antioxidants 2019, 8 (11), 556.
(10) Wo, Y.; Brisbois, E. J.; Bartlett, R. H.; Meyerhoff, M. E. Recent Advances in Thromboresistant and Antimicrobial Polymers for Biomedical Applications: Just Say Yes to Nitric Oxide (NO). Biomater. Sci. 2016, 4 (8), 1161-1183.
(11) DiBlasi, R. M.; Myers, T. R.; Hess, D. R. Evidence-Based Clinical Practice Guideline: Inhaled Nitric Oxide for Neonates With Acute Hypoxic Respiratory Failure. Respir. Care 2010, 55 (12), 1717-1745.
(12) Yang, T.; Zelikin, A. N.; Chandrawati, R. Progress and Promise of Nitric Oxide-Releasing Platforms. Adv. Sci. 2018, 5 (6), 1701043.
(13) Riccio, D. A.; Schoenfisch, M. H. Nitric Oxide Release: Part I. Macromolecular Scaffolds. Chem. Soc. Rev. 2012, 41 (10), 3731.
(14) Chug, M. K.; Brisbois, E. J. Recent Developments in Multifunctional Antimicrobial Surfaces and Applications toward Advanced Nitric Oxide-Based Biomaterials. ACS Maier. Au 2022, 2 (5), 525-551.
(15) Poh, W. H.; Rice, S. A. Recent Developments in Nitric Oxide Donors and Delivery for Antimicrobial and Anti-Biofilm Applications. Molecules 2022, 27 (3), 674.
(16) Winchester, S.; John, S.; Jabbar, K.; John, T. Clinical Efficacy of Nitric Oxide Nasal Spray (NONS) for the Treatment of Mild COVID-19 Infection. J. Infect. 2021, 83 (2), 237-279.
(17) Schanuel, F. S.; Raggio Santos, K. S.; Monte-Alto-Costa, A.; de Oliveira, M. G. Combined Nitric Oxide-Releasing Poly(Vinyl Alcohol) Film/F127 Hydrogel for Accelerating Wound Healing. Colloids Surf, B 2015, 130, 182-191.
(18) Parent, M.; Boudier, A.; Perrin, J.; Vigneron, C.; Maincent, P.; Violle, N.; Bisson, J.-F.; Lartaud, I.; Dupuis, F. In Situ Microparticles Loaded with S-Nitrosoglutathione Protect from Stroke. PLoS One 2015, 10 (12), e0144659.
(19) Wu, W.; Gaucher, C.; Diab, R.; Fries, I.; Xiao, Y.-L.; Hu, X.-M.; Maincent, P.; Sapin-Minet, A. Time Lasting S-Nitrosoglutathione Polymeric Nanoparticles Delay Cellular Protein S-Nitrosation. Eur. J. Pharm. Biopharm. 2015, 89, 1-8.
(20) Kumar, R.; Massoumi, H.; Chug, M. K.; Brisbois, E. J. S-Nitroso-N-Acetyl-l-Cysteine Ethyl Ester (SNACET) Catheter Lock Solution to Reduce Catheter-Associated Infections. ACS Appl. Mater. Interfaces 2021, 13 (22), 25813-25824.
(21) Ashcraft, M.; Douglass, M.; Garren, M.; Mondal, A.; Bright, L. E.; Wu, Y.; Handa, H. Nitric Oxide-Releasing Lock Solution for the Prevention of Catheter-Related Infection and Thrombosis. ACS Appl. Bio Mater. 2022, 5 (4), 1519-1527.
(22) Mihu, M. R.; Cabral, V.; Pattabhi, R.; Tar, M. T.; Davies, K. P.; Friedman, A. J.; Martinez, L. R.; Nosanchuk, J. D. Sustained Nitric Oxide-Releasing Nanoparticles Interfere with Methicillin-Resistant Staphylococcus Aureus Adhesion and Biofilm Formation in a Rat Central Venous Catheter Model. Antimicrob. Agents Chemother. 2016, 61 (1), e02020-16.
(23) Carlsson, S.; Weitzberg, E.; Wiklund, P.; Lundberg, J. O. Intravesical Nitric Oxide Delivery for Prevention of Catheter-Associated Urinary Tract Infections. Antimicrob. Agents Chemother. 2005, 49 (6), 2352-2355.
(24) Ahmadi, M. S.; Lee, H. H.; Sanchez, D. A.; Friedman, A. J.; Tar, M. T.; Davies, K. P.; Nosanchuk, J. D.; Martinez, L. R. Sustained Nitric Oxide-Releasing Nanoparticles Induce Cell Death in Candida albicans Yeast and Hyphal Cells, Preventing Biofilm Formation In Vitro and in a Rodent Central Venous Catheter Model. Antimicrob. Agents Chemother. 2016, 60 (4), 2185-2194.
(25) Kishikawa, H.; Ebberyd, A.; Römling, U.; Brauner, A.; Liithje, P.; Lundberg, J. O.; Weitzberg, E. Control of Pathogen Growth and Biofilm Formation Using a Urinary Catheter That Releases Antimicrobial Nitrogen Oxides. Free Radicals Biol. Med. 2013, 65, 1257-1264.
(26) Vesey, C. J.; Cole, P. V. Blood Cyanide and Thiocyanate Concentrations Produced by Long-term Therapy with Sodium Nitroprusside. Br. J. Anaesth. 1985, 57 (2), 148-155.
(27) Hrabie, J. A.; Keefer, L. K. Chemistry of the Nitric Oxide-Releasing Diazeniumdiolate (“Nitrosohydroxylamine”) Functional Group and Its Oxygen-Substituted Derivatives. Chem. Rev. 2002, 102 (4), 1135-1154.
(28) Product Information: S-Nitroso-N-acetyl-DL-penicillamine.
(29) Broniowska, K. A.; Diers, A. R.; Hogg, N. S-Nitrosoglutathione. Biochim. Biophys. Acta 2013,1830 (5), 3173-3181.
(30) Molloy, J.; Martin, J. F.; Baskerville, P. A.; Fraser, S. C. A.; Markus, H. S. S-Nitrosoglutathione Reduces the Rate of Embolization in Humans. Circulation 1998, 98 (14), 1372-1375.
(31) Colagiovanni, D. B.; Borkhataria, D.; Looker, D.; Schuler, D.; Bachmann, C.; Sagelsdorff, P.; Honarvar, N.; Rosenthal, G. J. Preclinical 28-Day Inhalation Toxicity Assessment of S-Nitrosoglutathione in Beagle Dogs and Wistar Rats. Int. J. Toxicol. 2011, 30 (5), 466-477.
(32) Hornyak, I.; Pankotai, E.; Kiss, L.; Lacza, Z. Current Developments in the Therapeutic Potential of S-Nitrosoglutathione, an Endogenous NO-Donor Molecule. Curr. Pharm. Biotechnol. 2011, 12 (9), 1368-1374.
(33) Smith, J. N.; Dasgupta, T. P. Kinetics and Mechanism of the Decomposition of S-Nitrosoglutathione by 1-Ascorbic Acid and Copper Ions in Aqueous Solution to Produce Nitric Oxide. Nitric Oxide 2000, 4 (1), 57-66.
(34) de Oliveira, M. G.; Shishido, S. M.; Seabra, A. B.; Morgon, N. H. Thermal Stability of Primary S-Nitrosothiols: Roles of Autocatalysis and Structural Effects on the Rate of Nitric Oxide Release. J. Phys. Chem. A 2002, 106 (38), 8963-8970.
(35) de Souza, G. F. P.: Denadai, J. P.; Picheth, G. F.; de Oliveira, M. G. Long-Term Decomposition of Aqueous S-Nitrosoglutathione and S-Nitroso-N-Acetylcysteine: Influence of Concentration, Temperature, PH and Light. Nitric Oxide 2019, 84, 30-37.
(36) Wink, D. A.; Mitchell, J. B. Chemical Biology of Nitric Oxide: Insights into Regulatory, Cytotoxic, and Cytoprotective Mechanisms of Nitric Oxide. Free Radicals Biol. Med. 1998, 25 (4), 434-456.
(37) Thomas, D. D.; Ridnour, L. A.; Isenberg, J. S.; Flores-Santana, W.; Switzer, C. H.; Donzellie, S.; Hussain, P.; Vecoli, C.; Paolocci, N.; Ambs, S.; Colton, C.; Harris, C.; Roberts, D. D.; Wink, D. A. The Chemical Biology of Nitric Oxide. Implications in Cellular Signaling. Free Radicals Biol. Med. 2008, 45 (1), 18-31.
(38) Lee, J.; Kwak, D.; Kim, H.; Kim, J.; Hlaing, S. P.; Hasan, N.; Cao, J.; Yoo, J.-W. Nitric Oxide-Releasing S-Nitrosoglutathione-Conjugated Poly(Lactic-Co-Glycolic Acid) Nanoparticles for the Treatment of MRSA-Infected Cutaneous Wounds. Pharmaceutics 2020, 12 (7), 618.
(39) Wu, W.; Gaucher, C.; Fries, I.; Hu, X.; Maincent, P.; Sapin-Minet, A. Polymer Nanocomposite Particles of S-Nitrosoglutathione: A Suitable Formulation for Protection and Sustained Oral Delivery. Int. J. Pharm. 2015, 495 (1), 354-361.
(40) Diab, R.; Virriat, A. S.; Ronzani, C.; Fontanay, S.; Grandemange, S.; Elaissari, A.; Foliguet, B.; Maincent, P.; Leroy, P.; Duval, R. E.; Rihn, B. H.; Joubert, O. Elaboration of Sterically Stabilized Liposomes for S-Nitrosoglutathione Targeting to Macrophages. J. Biomed. Nanotechnol. 2016, 12 (1), 217-230.
(41) Safar, R.; Ronzani, C.; Diab, R.; Chevrier, J.; Bensoussan, D.; Grandemange, S.; Le Faou, A.; Rihn, B. H.; Joubert, O. Human Monocyte Response to S-Nitrosoglutathione-Loaded Nanoparticles: Uptake, Viability, and Transcriptome. Mol. Pharmaceutics 2015, 12 (2), 554-561.
(42) Jacob, S.; Nair, A. B. Cyclodextrin Complexes: Perspective from Drug Delivery and Formulation. Drug Dev. Res. 2018, 79 (5), 201-217.
(43) Khan, D. N. A.; Khan, N. A.; Durakshan, M. Cyclodextrin: An Overview. Int. J. Bioassays 2013, 2 (6), 858-865.
(44) Hirayama, F. Cyclodextrin-Based Controlled Drug Release System. Adv. Drug Delivery Rev. 1999, 36 (1), 125-141.
(45) Davis, M. E.; Brewster, M. E. Cyclodextrin-Based Pharmaceutics: Past, Present and Future. Nat. Rev. Drug Discov. 2004, 3 (12), 1023-1035.
(46) Umemura, M.; Ueda, H.; Tomono, K.; Nagai, T. Effect of Diethyl-Beta-Cyclodextrin on the Release of Nitroglycerin from Formulations. Drug Des. Deliv. 1990, 6 (4), 297-310.
(47) Centkowska, K.; Sznitowska, M. Modified-Release Ointment with Nitroglycerin p-Cyclodextrin Inclusion Complex for Treatment of Anal Fissures. J. Pharm. Pharmacol. 2013, 65 (10), 1463-1472.
(48) Bauer, J. A.; Fung, H.-L. Chemical Stabilization of a Vasoactive S-Nitrosothiol with Cyclodextrins Without Loss of Pharmacologic Activity. Pharm. Res. 1991, 8 (10), 1329-1334.
(49) Bookstaver, B.; Justo, J. A. Antibiotic Lock Therapy: Review of Technique and Logistical Challenges. Infect. Drug Resist. 2014, 343.
(50) Piras, L.; Theodossiou, T. A.; Manouilidou, M. D.; Lazarou, Y. G.; Sortino, S.; Yannakopoulou, K. S-Nitroso-o-Cyclodextrins as New Bimodal Carriers: Preparation, Detailed Characterization, Nitric-Oxide Release, and Molecular Encapsulation. Chem. Asian J. 2013, 8 (11), 2768-2778.
(51) Jin, H.; Yang, L.; Ahonen, M. J. R.; Schoenfisch, M. H. Nitric Oxide-Releasing Cyclodextrins. J. Am. Chem. Soc. 2018, 140 (43), 14178-14184.
(52) Melvin, A. C.; Jones, W. M.; Lutzke, A.; Allison, C. L.; Reynolds, M. M. S-Nitrosoglutathione Exhibits Greater Stability than S-Nitroso-N-Acetylpenicillamine under Common Laboratory Conditions: A Comparative Stability Study. Nitric Oxide 2019, 92, 18-25.
(53) Williams, D. L. H. The Chemistry of S-Nitrosothiols. Acc. Chem. Res. 1999, 32 (10), 869-876.
(54) Grimme, S.; Hansen, A.; Ehlert, S.; Mewes, J.-M. R2SCAN-3c: A “Swiss Army Knife” Composite Electronic-Structure Method. J. Chem. Phys. 2021, 154 (6), 064103.
(55) Carter-Fenk, K.; Herbert, J. M. Appraisal of Dispersion Damping Functions for the Effective Fragment Potential Method. Molecular Physics 2022, 0 (0), e2055504.
(56) Villot, C.; Ballesteros, F.; Wang, D.; Lao, K. U. Coupled Cluster Benchmarking of Large Noncovalent Complexes in L7 and S12L as Well as the C60 Dimer, DNA-Ellipticine, and HIV-Indinavir. J. Phys. Chem. A 2022, 126 (27), 4326-4341.
(57) Neese, F.; Wennmohs, F.; Becker, U.; Riplinger, C. The ORCA Quantum Chemistry Program Package. J. Chem. Phys. 2020, 152 (22), 224108.
(58) Li, W.; Wang, D.; Lao, K. U.; Wang, X. Buffer Concentration Dramatically Affects the Stability of S-Nitrosothiols in Aqueous Solutions. Nitric Oxide 2022, 118, 59-65.
(59) Masters, K. S. B.; Leibovich, S. J.; Belem, P.; West, J. L.; Poole-Warren, L. A. Effects of Nitric Oxide Releasing Poly(Vinyl Alcohol) Hydrogel Dressings on Dermal Wound Healing in Diabetic Mice. Wound Repair Regen. 2002, 10 (5), 286-294.
(60) Kim, J.; Francis, D. M.; Sestito, L. F.; Archer, P. A.; Manspeaker, M. P.; O'Melia, M. J.; Thomas, S. N. Thermosensitive Hydrogel Releasing Nitric Oxide Donor and Anti-CTLA-4 Micelles for Anti-Tumor Immunotherapy. Nat. Commun. 2022, 13 (1), 1479.
(61) Zhou, Y.; Tan, J.; Dai, Y.; Yu, Y.; Zhang, Q.; Meyerhoff, M. E. Synthesis and Nitric Oxide Releasing Properties of Novel Fluoro S-Nitrosothiols. Chem. Commun. 2019, 55 (3), 401-404.
(62) Baciu, C.; Gauld, J. W. An Assessment of Theoretical Methods for the Calculation of Accurate Structures and SN Bond Dissociation Energies of S-Nitrosothiols (RSNOs). J. Phys. Chem. A 2003, 107 (46), 9946-9952.
(63) Tsikas, D.; Sandmann, J.; Lueβen, P.; Savva, A.; Rossa, S.; Stichtenoth, D. O.; Frölich, J. C. S-Transnitrosylation of Albumin in Human Plasma and Blood in Vitro and in Vivo in the Rat. Biochim. Biophys. Acta, Protein Structure and Molecular Enzymology 2001, 1546 (2), 422-434.
(64) Wang, X.; Jolliffe, A.; Carr, B.; Zhang, Q.; Bilger, M.; Cui, Y.; Wu, J.; Wang, X.; Mahoney, M.; Rojas-Pena, A.; J. Hoenerhoff, M.; Douglas, J.; H. Bartlett, R.; Xi, C.; L. Bull, J.; E. Meyerhoff, M. Nitric Oxide-Releasing Semi-Crystalline Thermoplastic Polymers: Preparation, Characterization and Application to Devise Anti-Inflammatory and Bactericidal Implants. Biomater. Sci. 2018, 6 (12), 3189-3201.
(65) Chug, M. K.; Feit, C.; Brisbois, E. J. Increasing the Lifetime of Insulin Cannula with Antifouling and Nitric Oxide Releasing Properties. ACS Appl. Bio Mater. 2019, 2 (12), 5965-5975.
(66) Tandon, M.; Wu, W.; Moore, K.; Winchester, S.; Tu, Y.-P.; Miller, C.; Kodgule, R.; Pendse, A.; Rangwala, S.; Joshi, S. SARS-CoV-2 Accelerated Clearance Using a Novel Nitric Oxide Nasal Spray (NONS) Treatment: A Randomized Trial. Lancet Reg. Health Southeast Asia 2022, 3, 100036.
(67) Sung, Y. C.; Jin, P. R.; Chu, L. A.; Hsu, F. F.; Wang, M. R.; Chang, C. C.; Chiou, S. J.; Qiu, J. T.; Gao, D. Y.; Lin, C. C.; Chen, Y. S.; Hsu, Y. C.; Wang, J.; Wang, F. N.; Yu, P. L.; Chiang, A. S.; Wu, A. Y. T.; Ko, J. J. S.; Lai, C. P. K.; Lu, T. T.; Chen, Y. Delivery of Nitric Oxide with a Nanocarrier Promotes Tumour Vessel Normalization and Potentiates Anti-Cancer Therapies. Nat. Nanotechnol. 2019, 14 (12), 1160-1169.
(68) CDC: Current HAI Progress Report.
(69) Nguyen, D. B.; Shugart, A.; Lines, C.; Shah, A. B.; Edwards, J.; Pollock, D.; Sievert, D.; Patel, P. R. National Healthcare Safety Network (NHSN) Dialysis Event Surveillance Report for 2014. Clin. J. Am. Soc. Nephrol. 2017, 12 (7), 1139-1146.
(70) Liu, Y.; Zhang, A.-Q.; Cao, L.; Xia, H.-T.; Ma, J.-J. Taurolidine Lock Solutions for the Prevention of Catheter-Related Bloodstream Infections: A Systematic Review and Meta-Analysis of Randomized Controlled Trials. PLoS One 2013, 8 (11), e79417.
(71) Mermel, L. A. What Is The Predominant Source of Intravascular Catheter Infections?Clin. Infect. Dis. 2011, 52 (2), 211-212.
(72) Ren, H.; Bull, J. L.; Meyerhoff, Mark. E. Transport of Nitric Oxide (NO) in Various Biomedical Grade Polyurethanes: Measurements and Modeling Impact on NO Release Properties of Medical Devices. ACS Biomater. Sci. Eng. 2016, 2 (9), 1483-1492.
(73) de Mel, A.; Murad, F.; Seifalian, A. M. Nitric Oxide: A Guardian for Vascular Grafts?Chem. Rev. 2011, 111 (9), 5742-5767.
(74) Goossens, G. A. Flushing and Locking of Venous Catheters: Available Evidence and Evidence Deficit. Nurs. Res. Pract. 2015, 2015, e985686.
(75) Sievert, D. M.; Ricks, P.; Edwards, J. R.; Schneider, A.; Patel, J.; Srinivasan, A.; Kallen, A.; Limbago, B.; Fridkin, S.; National Healthcare Safety Network (NHSN) Team and Participating NHSN Facilities. Antimicrobial-Resistant Pathogens Associated with Healthcare-Associated Infections: Summary of Data Reported to the National Healthcare Safety Network at the Centers for Disease Control and Prevention, 2009-2010. Infect. Control. Hosp. Epidemiol. 2013, 34 (1), 1-14.
(76) Williams, D. E.; Boon, E. M. Towards Understanding the Molecular Basis of Nitric Oxide-Regulated Group Behaviors in Pathogenic Bacteria. J. Innate Immun. 2019, 11 (3), 205-215.
(77) Beathard, G. A.; Urbanes, A. Infection Associated with Tunneled Hemodialysis Catheters. Semin. Dial. 2008, 21 (6), 528-538.
(78) Fang, F. C. Perspectives Series: Host/Pathogen Interactions. Mechanisms of Nitric Oxide-Related Antimicrobial Activity. J. Clin. Invest. 1997, 99 (12), 2818-2825.
(79) Brady, B.; Li, W.; Farooque, N.; Ehrhardt, C.; Meyerhoff, M. E.; Wang, X. S-Nitrosothiol-Impregnated Silicone Catheter for Colorimetric Sensing of Indole and E. Coli: Toward On-Body Detection of Urinary Tract Infections. ACS Sens. 2022, 7 (6), 1712-1719.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

What is claimed is:

1. A composition, comprising guest/host inclusion complexes, each complex comprising S-nitrosoglutathione (GSNO) and at least one substituted or unsubstituted cyclodextrin.

2. The composition of claim 1, wherein the at least one substituted or unsubstituted cyclodextrin comprises two or more types of cyclodextrin.

3. The composition of claim 1, wherein the at least one substituted or unsubstituted cyclodextrin is not S-nitrosylated.

4. The composition of claim 1, wherein the at least one substituted or unsubstituted cyclodextrin is a substituted cyclodextrin and is an acetylated, alkylated, hydroxyalkylated, methylated, hydroxyethylated, or hydroxypropylated cyclodextrin.

5. The composition of claim 1, wherein the at least one substituted or unsubstituted cyclodextrin is selected from the group consisting of alpha cyclodextrin, gamma cyclodextrin, hydroxypropyl beta cyclodextrin, acetyl beta cyclodextrin, methyl alpha cyclodextrin, methyl beta cyclodextrin, 2-hydroxypropyl alpha cyclodextrin, 2-hydroxypropyl gamma cyclodextrin, 2-hydroxyethyl beta cyclodextrin, and 2,3,6-tri-o-methyl beta cyclodextrin.

6. The composition of claim 1, wherein the at least one substituted or unsubstituted cyclodextrin is not co-complexed with a polymer.

7. The composition of claim 1, wherein a molar ratio of GSNO to cyclodextrin is from 1:10 to 10:1.

8. The composition of claim 1, wherein the composition is in a liquid solution or suspension dosage form.

9. The composition of claim 1, wherein the composition is in a gel dosage form.

10. The composition of claim 1, wherein the composition is in a dried dosage form.

11. A method of delivering nitric oxide to a subject in need thereof, comprising administering an effective amount of the composition of claim 1 to the subject.

12. A method of inhibiting microbial growth on a surface, comprising contacting the surface with the composition of claim 1.

13. The method of claim 12, wherein the surface is on a catheter and wherein the catheter is at least partially filled with a solution containing the composition.
